(19) United States
(12) Patent Application Publication
(10) Pub. No.: US 2019/0209288 A1
(43) Pub. Date: Jul. 11, 2019

(54) SYSTEM FOR THE INTRAOSSEOUS ATTACHMENT OF A FLEXIBLE WIRE INTENDED FOR ANCHORING LIGAMENT TISSUE TO BONE

(71) Applicant: MEDACTA INTERNATIONAL S.A., Castel San Pietro (CH)

(72) Inventor: Michel Collette, Bruxelles (BE)

(21) Appl. No.: 15/754,906

(22) PCT Filed: Aug. 26, 2016
(86) PCT No.: PCT/EP2016/070265

§ 371 Date: Feb. 23, 2018

(30) Foreign Application Priority Data
Aug. 26, 2015 (BE) 2015/5539
Jan. 18, 2016 (EP) PCT/EP2016/050929

(51) Int. Cl. A61F 2/08; A61B 17/04; A61B 17/06

(52) U.S. Cl. CPC A61F 2/0811; A61B 17/0401; A61B 17/06; A61F 2002/0852; A61B 2017/044; A61B 2017/06176; A61F 2002/0882; A61F 2002/0858

(57) ABSTRACT

The invention concerns a device for attaching soft tissue to bone tissue, e.g. a broken ligament or ligament graft (1), comprising a flexible wire (10) provided with several corpuscles (9, 9′, 9″) fixed along the length of same, and a screw (8) capable of engaging with said corpuscles of the wire (10) when screwed into a bone tunnel (4) by blocking them between the screw pitch and the bone tissue. The wire is capable of forming a free loop (2) for receiving said soft tissue, e.g. a ligament graft, the two branches of said loop being provided with said corpuscles. The wire can also be provided with a closed loop or a crimped needle. The screw is a cannulated screw for receiving a guide pin (6).

### BACKGROUND OF THE INVENTION

The anchoring of capsular or ligament soft tissues to bone constitutes one of the most frequent surgical requirements after a sports injury. The attachment of a ligament graft in bone tissue in view of reconstructing the broken anterior cruciate ligament is among the most frequent (but not exclusive) examples of this type of surgical procedure. This application has been chosen for disclosing the present invention but it is clear that it can equally be applied to other anatomical structures.

The surgical reconstruction of a broken anterior cruciate ligament (ACL) is most often performed nowadays by means of a tendon graft taken from either the patellar tendon or the tendons of the inner posterior hamstrings of the thigh and of the knee, the semitendinosus and gracilis muscle (ST-GRA).

Although patellar tendon grafts are considered to be more effective from a mechanical viewpoint, ST-GRA grafts are increasingly common as the adverse secondary effects connected with their harvesting are generally less severe than with patellar tendon grafts.

Conventionally ST-GRA grafts are constructed by folding each of the two tendons harvested (semitendinosus and gracilis muscle) onto themselves at their centre so as to obtain a four-strand graft measuring approximately 12 to 15 cm. As the length of the cruciate ligament to be constructed is only about 3 cm, it is clear that 70 to 80% of the tendon substance harvested is used exclusively for the attachment of the useful length of the graft (3 cm intra-articular).

The Applicant has previously described a new system that was the subject matter of patent applications (see publications PCT WO2004/045465 and WO2007/147634), allowing the same operation to be performed by harvesting a single tendon, the semitendinosus and folding it onto itself twice to obtain, as in conventional techniques, a four-strand tendon graft, but with the inevitable consequence of obtaining a smaller graft (about 5 or 6 cm). The saving of tendon tissue thus created undoubtedly constitutes progress in relation to conventional techniques, but it induces new technical difficulties related to the attachment of this short graft to the bone. In fact, the reduced quantity of ligament tissue introduced into the bone no longer allows conventional attachment processes to be used such as the compression of the graft in the bone tunnel by a clamping screw introduced between the graft and the inner wall of the bone tunnel (interference screw) or even the suspension of the ligament eyelet by a solid axle-shaped member transfixing the graft and attached into the bone by approaching the bone tunnel, perpendicularly to the axis of the graft.

The application of a short graft therefore imposes the use of synthetic bridges for extending the graft at both ends thereof and connecting it to its attaching member.

A first process consists of anchoring the attaching member at cortical bone level, i.e. at the exit of the bone tunnel in which the graft is located. The cortical bone is undoubtedly the most resistant bone area therefore offering the anchoring system extremely high tensile strength (more than 1000 Newtons). However, the shorter the graft, the longer the synthetic bridge must be before reaching the cortical level where the attaching member is located. Yet, it has been clearly and repeatedly shown experimentally, on one hand, that the elasticity of the assembly increases in proportion to the length of the synthetic bridge and, on the other hand, that such elasticity has a negative effect on the mechanical properties of the system. In fact, the elasticity of the synthetic bridges promotes axial or transverse micro movements inside the bone tunnel which inevitably implies a slackening of the tension of the graft and progressively an enlargement of the bone tunnels that house it.

A second process consists of anchoring the attaching member in proximity to the graft thus reducing as much as possible the length of the synthetic bridge between the graft and the attaching member and therefore the overall elasticity of the assembly. This system describes the suspension of the short graft by two fabric strips (polyethylene terephthalate) attached to the actual inside of the bone tunnel by a special screw called a TLS (Tape Locking Screw) introduced from the outside to the inside, in the direction of the graft and practically until its contact which effectively counteracts the elasticity of the fabric bridge.

Although the TLS has now taken off considerably throughout the world, (more than 30,000 cases performed to date), it implies some residual drawbacks that have in part been described in patent application WO2013/1 39985 entitled “Système de fixation d'une greffe ligamentaire”. Clinical experience has, in fact, made it possible to identify many factors susceptible to affecting the quality of the final result:
the quality of the bone in which the anchoring is performed. The softer the bone, the less solid the attachment will be.
The quality of screwing: excess or lack of penetration of the screw into the bone tunnel is susceptible to affecting the solidity of the assembly. The same applies if the axis of penetration of the screw diverges in relation to the axis of the bone tunnel in which the strips pass.
The quality of the ribbon deployment inside the bone tunnel remains an uncontrollable factor to be managed, also susceptible to affecting the performance of the screwing of the ribbon.
To allow the passage of strips in the bone tunnels, the borehole diameter of these tunnels must be at least 4.5 mm. Although this already constitutes progress in relation to conventional techniques which require tunnels to be bored at the diameter of the graft (8 to 10 mm), this borehole diameter requirement somewhat affects the minimally invasive nature that this technique seeks to achieve.
After the boring of the tunnel, the TLS technique imposes tapping the entry of the bone tunnel to prepare the housing of the screw. Although this is relatively simple to perform, this task still constitutes an extra step in relation to other techniques where this technical task does not exist, hence extending the operating time which is ideally to be as short as possible.—Finally, the relatively large volume of the TLS screw somewhat compromises the saving of bone tissues which the method aims to achieve and therefore reduces its minimally invasive nature. This drawback appears to be particularly marked in the event of reconstructing anterior cruciate ligaments in children where the bone structures are small. Patent WO2013/1 39985 (TLS 3 system) therefore proposes a system that aims to correct various weaknesses of the TLS system:
The replacement of ribbons by a suspension wire allows the diameter of the bone tunnels necessary for its passage in the bone to be reduced.
The use of a pin for clamping the wire at its centre allows the problems to be solved connected with the quality of the surrounding bone, the quality of screwing and problems connected with the deployment of the ribbon inside the bone tunnel.

However, like the initial TLS system, the housing of the clamping pin requires the creation of a specific bone seat which burdens the technical realisation of the operation and has a negative effect on the minimally invasive nature of the procedure, which is one of the main objectives of this new technique.

The present invention conserves the use of a suspension wire (whose advantages in relation to suspension strips have been described above), conserves the possibility of counteracting the elasticity of the wire as in the original TLS technique by introducing the attaching member into the bone tunnel practically until contact of the graft, but suppresses the need to create a specific bone seat intended to receive the attaching member.

The texture and size of the suspension strips used in the original TLS technique allowed a solid attachment to be obtained by blocking the ribbon between the fixing screw and the wall of the bone tunnel.

The same result cannot unfortunately be achieved by screwing a simple wire whose smooth and very reduced surface would cause exposure to the risk of slippage due to a significantly lower tensile constraint than that required for attaching a ligament (600 to 900 Newtons). On the other hand, by attaching on the wire several small solid nodules, a flexible corpuscular wire is obtained, which is favourable for intraosseous attachment by interference with the relief of a simple screw introduced into the same bone tunnel.

Additional Comparative Discussion of the Closest Prior Art

Research into the prior art has highlighted the existence of a patent application WO 2012/058301 that already describes the use of a thin wire equipped with several corpuscules, notably intended for attachment of broken ligament tissue onto the bone. The locking mechanism and the mechanical effect obtained are therefore entirely different.

In fact, in the system of application WO 2012/058301, the filament is displaced in the direction of the locking member. This means that the positioning of the corpuscular wire and its locking in the bone tunnel take place simultaneously: as the wire is displaced in the tunnel, it is automatically locked in its new position, without the possibility to return backwards. In practice, if the surgeon realises that the graft is too soft, the locking system as described in this invention, no longer allows him to loosen the suspension wire since, by definition, the wire is locked in only one displacement direction.

With the device according to the present invention, as can be more clearly understood from the following description, the positioning of the wire in the tunnel on one hand and the locking process of the wire on the other hand, constitute two strictly independent steps from each other. In practice, the surgeon starts by freely positioning the graft in the bone tunnel acting through traction on the wire, if he wishes to stretch the graft, or by traction on the graft if he wishes to loosen it, and it is only after adjusting the positioning and tension of the graft that he inserts the locking screw which is displaced in the tunnel, along the wire which, itself, remains perfectly stationary. If even after inserting the screw, the surgeon notices a mistake in the adjustment of the tension, according to the present invention, the device still allows the attaching member to be unscrewed, the tension adjustment to be performed and the locking screw to be inserted again. Furthermore, the necessary length of the suspension wire corresponds to the distance between the end of the ligament to be attached and the support zone of the locking member. In the example of our short cruciate ligament graft, the end of the graft is in proximity to the intra-articular entry orifice of the bone tunnel when the locking member needs to be placed at cortical level, i.e. at the exit of this same tunnel, as also illustrated by FIG. 12 of the original description. This process would therefore produce a mechanically unfavourable suspension system as has already been explained above, comprising a long flexible bridge, therefore elastic, between the graft, located at the intra-articular entry of the bone tunnel, and the locking member, located at cortical level, i.e. at the exit of the bone tunnel.

On the other hand, in the present device or system, the locking screw progresses from the outside to the inside, in the direction of the graft and locks the wire practically until contact with the graft, thus counteracting the potential elasticity of the suspension system.

Finally, since the anchorage is guaranteed by the locking of two corpuscules through the two one-way orifices, the pull-out resistance of the tissue organ thus attached is limited to the cumulative pull-out resistance of two corpuscules on the wire.

In the system according to the present invention, the locking of the corpuscules, and therefore of the wire, takes place inside the bone tunnel itself by the insertion into this tunnel of a screw whose relief interacts with the relief of all the corpuscules. Various different types of interaction may be envisaged, essentially depending on the manufacturing method of the corpuscular wire and the type of screw used.

Another patent application WO 2007/070024 also describes a corpuscular wire system allowing anchorage to be obtained through a screw.

This patent document obviously describes the use of a corpuscular filament but which only acts if it is inserted in a large number into a bone cavity where expansion is to be caused by packing. The compacting of the innumerable corpuscules joined to each other by filaments thus creates a dense and sufficiently compact environment that in order to be created requires anchorage through a screw. This invention therefore describes a means allowing a screw to be anchored into a bone cavity previously packed with multiple corpuscular wires.

On the other hand, our invention describes a device that aims to anchor not a screw but a single corpuscular wire into a thin bone tunnel, thanks to a screw.

There are multiple advantages of the present system with respect to the system of the prior art:
the adjustment of the positioning of the graft and its tensioning are performed completely freely and independently from the locking process which only takes place secondarily, to “set” the position chosen by the surgeon. In the event of problems, the screw can also be removed, then inserted again after the tension has been readjusted according to the surgeon's wishes.
the screw locks all the corpuscules attached to the wire and thus counteracts its elasticity practically until contact of the graft (as in the original TLS system).
the screw may be introduced directly into the tunnel, without the preparation of a special housing (as the TLS system requires).
the corpuscules being attached in series to the wire, the pull-out resistance of the graft corresponds to the cumulative pull-out resistance of all the corpuscules secured by the screw. If, for example, the pull-out resistance of a corpuscule on the wire is 10 kg, the resistance of a wire comprising 10 secured corpuscules will be about 100 kg.
while maintaining the highly performing mechanical properties, the implementation of this system takes place through a particularly minimally invasive technique (extremely small bore tunnels, wire and screw).
Although in the device and technical process according to the invention the screw is inserted from the outside to the inside, the small gauge of this screw can, if necessary, allow it to be inserted from the inside outwards, as in conventional in-out ligamentoplasty techniques.

### SUMMARY OF THE INVENTION

The present invention proposes a system that allows a ligament graft to be suspended and attached through bone tunnels reduced to their smallest size (2-3 mm) and without having to bore a specific bone seat intended to receive the attaching member.

This system comprises a very solid wire onto which several solid corpuscules are attached, and a locking member comprising a screw inserted into the same bone tunnel parallel to the corpuscular wire. The suspension wire can be comprised of a single filament which finishes at one of its ends with a closed loop intended to suspend the graft. The suspension wire can also finish with a crimped needle which allows the lacing of the end of the ligament to be re-attached to the bone. It can also be folded onto itself to form a natural loop intended to suspend the graft, each suspension strand being able to be equipped with corpuscules. The wire bearing the graft is adapted to be pulled into a bone tunnel whose diameter is slightly larger than the diameter of the wire which, according to the application, can vary between 0.5 and 2 mm. The locking of the wire (and therefore of the underlying graft) is obtained by the introduction into the bone channel itself of a conventional bone screw, routed in the bone tunnel parallel to the wire. If necessary for the surgical technique (e.g. ligamentoplasty), it is possible to use a screw that is cannulated in its centre according to its longitudinal axis, commonly used in orthopaedics, so as to be able to thread it onto a fine guide pin, previously arranged in the bone tunnel and intended to guide the path of the screw inside the tunnel with precision. The conventional method of realisation consists of introducing the screw from the outside to the inside, in the direction of the graft but, according to the circumstances and the objective to be reached, there is nothing against the insertion of the screw through the ligament tissue itself and its progression from the inside to the outside, moving away from the graft, in the same way as in conventional in-out ligamentoplasty techniques. According to the type of corpuscules used, the locking is either obtained by the driving and embedding of the corpuscules into the bone wall, or by trapping the corpuscules in the space located between two adjacent threads, between the core of the screw and the nearby bone, or by a mechanism that combines the two effects.

In a first method of realisation, the corpuscules may be oblong and have a larger longitudinal dimension than distance of the screw pitch. In this case, the insertion of the screw into the tunnel causes the expulsion of the corpuscular filament outside the body of the screw, the corpuscules actually being embedded in the nearby bone tissue. The shape of the corpuscule can also be optimised to promote this intraosseous penetration.

In a second method of realisation, the distance between the corpuscules and their size are adjusted so that they become lodged in the space located between the threads of the screw, which therefore create a practically impassable obstacle to the sliding of the corpuscules. In fact, they are thus trapped, above and below by the thread of the screw and laterally, by the rigid wall of the bone tunnel.

The two modes can be advantageously combined through the use of corpuscules whose dimensions simultaneously allow the enclosure between the threads of the screw (by the adjustment of the inter-corpuscular distance) and the protrusion into the bone tissue (by the adjustment of their shape and diameter) (see below: description of the corpuscules).

Whatever method of realisation is used, there are shape and size requirements of the screw-corpuscule pair that constitute an essential condition for the proper operation of the system described.

It is, for example, important that the screw has a conical shape, mainly in the vicinity of its tip, which must be as fine as possible. In fact, it is necessary that as it progresses in the tunnel, the screw leaves the corpuscule enough space to be able to find its way between the tip thereof and the wall of the tunnel, so as to become embedded therein. It is easy to understand that a screw that is too large whose end occupies the whole diameter of the bone tunnel would cause the corpuscules encountered to be driven out of the tunnel, thus impeding any possibility of anchoring the wire in the tunnel.

To be effective, the interaction of the screw-corpuscule pair therefore requires the precise adjustment of its shape and the size of the screw in relation to the shape and size of the corpuscules, but also very precise adjustment of the size of its components in relation to the diameter of the bone tunnel, i.e. the dimensions of the auger used for boring the tunnels.

### DETAILED DESCRIPTION OF THE INVENTION

1. The Wire

To fulfil the requirements of the present invention, the suspension wire must be both very resistant to tensile forces, as rigid as possible and as small as possible to allow its insertion through bone tunnels of a minimal gauge.

Among the different bio-compatible textile materials available, Dyneema® fibre has mechanical characteristics that respond particularly well to the requirements of the system proposed. It is widely used in different domains that require very resistant ropes (water sports, parachuting, rock climbing, etc.) but also in various medical applications. It is a high density polyethylene fibre that is extremely resistant to tensile forces (15 times more resistant than steel wire) and with high rigidity (low elasticity) that corresponds exactly to the characteristics of the suspension system that we are aiming for. By way of example, a 1.5 to 2 mm wire can withstand loads of 200 to 300 kg which constitutes a resistance limit two to three times higher than the one necessary within the scope of the invention. This means that thanks to this type of fibres, it is possible to manufacture a wire with a very small diameter which, after the addition of the corpuscules will reach a final diameter of 1.5 to 2 mm being able to be introduced into a tunnel of 3 to 4 mm which constitutes significant process in relation to the TLS system which imposes a bone boring of 4.5 mm and all the more so in relation to conventional techniques which, as already mentioned, impose boring the bone tunnels with the diameter of the graft (8 to 10 mm).

The wire according to the invention is therefore comprised of an extremely resistant, small diameter, filament, onto which one or more corpuscules are to be attached, intended to counteract any possibility of slipping after the placement of the locking member (screw). Different methods of realisation allow the fulfilment of variations in circumstances and objectives to be met.

For example, the wire may be comprised of a single filament one end of which is folded back and fastened onto itself (by splicing, suturing, crimping, etc.) forming a free loop intended to suspend a ligament eyelet. Alternatively, the end of the wire can finish with a crimped needle for tacking the end of the ligament to be re-attached. Alternatively, the filament may be folded back onto itself thus creating a loop that can catch and suspend a ligament eyelet, each section of the wire situated on either side of the loop being able to be equipped with corpuscules. Certain applications may require the use of a simple corpuscular wire without the formation of a loop at the end thereof.

2. The Corpuscules

These are small solid members attached or integrated to the wire so as to create along said wire an anti-slip relief intended to interact with the relief of the locking member (screw). These corpuscules shall preferably be manufactured using bio-compatible composite material (e.g.: Peek, PLLA etc. . . . ) But any other solid and bio-compatible material may be used (e.g.: metal, etc.). The attachment of the corpuscule to the wire must be as solid as possible since it is the pull-out resistance of each corpuscule multiplied by the number of corpuscules that determines the total pull-out resistance of the wire itself and therefore of the ligament graft that it suspends.

The process for attaching the corpuscules onto the wire depends closely on the type of material chosen. Any manufacturing process (chemical, physical, mechanical, etc.) can be used as long as it allows solid attachment to the wire to be obtained.

The shape and size characteristics of the corpuscules play an essential role in the type of locking mechanism that is to be produced due to the effect of inserting the screw.

The creation of knots on the wire itself, arranged at a regular distance according to the size of the screw constitutes a particular method of realisation of the corpuscules.

Various types of locking mechanisms are possible according to the size and shape of the corpuscules:

a. An oblong corpuscule whose longitudinal dimension exceeds the distance separating 2 adjacent threads of the screw.

Its shape may be cylindrical but is more preferably rhomboid or trapezoid shaped so as to promote the penetration of the edges into the bone tissue. In the case of the figure, by penetrating into the bone tunnel, the screw will therefore push the corpuscules outside the axis of the bone tunnel, causing on one hand the embedding of each corpuscule inside the nearby bone tissue, and on the other hand, the impression of the thread of the screw on the wall of the corpuscule, the two effects counteracting the slipping at the corpuscule-bone interface level.

b. A short corpuscule whose diameter is adjusted to allow its enclosure between 2 adjacent threads of the screw.

Its shape is generally spherical but all other shapes having edges that promote its penetration into the bone are preferred if the size of the corpuscules permits this.

In the case of the figure, the screw will have the effect of trapping the corpuscule in the concavity situated between the threads of the screw, the corpuscule finding itself fixed on one side between the wall of the overlying and underlying thread and on the other side between the core of the screw and the nearby bone. This mechanism clearly requires precise adjustment between the dimensions of the screw and the length of the filament between each corpuscule during manufacturing.

3. The screw. According to the invention, after introducing the wire into the bone tissue and adjusting its tension according to the surgeon's wishes, it is sufficient to insert into the same tunnel a screw whose relief interacts with the corpuscules of the wire and completely counteracts its ability to slip.

In the example of ligamentoplasty, it is essential in order to obtain the locking effect, for the screw to travel in the bone tunnel according to a path that is rigorously parallel to the axis of the wire. This requirement is met by the use of a cannulated screw, commonly used in orthopaedic surgery. It is a screw that is hollow along its longitudinal axis, being able to slide on a fine guide pin inserted in advance into the bone tunnel, parallel to the corpuscular wire.

The use of such a guide pin further allows the placement of the screw through the skin and subcutaneous tissues, without having to surgically approach the bone surface where the exit of the bone tunnel is located.

It is underlined that the screw can produce its fixing effect:
either by simply driving the corpuscules into the bone tunnel wall (oblong corpuscules). In this case, the distance separating the threads of the screw is shorter than the length of the longitudinal axis of the corpuscules.
or by trapping the corpuscule in the space delimited by the wall of two adjacent threads, the core of the screw and the nearby bone. However, this configuration requires some very precise specifications during the manufacture of the screw and of the corpuscular wire. In fact, it is necessary that the length of the wire between each corpuscule is precisely adjusted so that each corpuscule can be lodged between the threads whilst maintaining suitable and identical tension from corpuscule to corpuscule. Therefore, there must be a very precise correspondence between the dimensions of the screw and the dimensions of the corpuscular wire. This configuration further requires the most projecting part of the thread of the screw to be sufficiently blunt to prevent any risk of the screw thread shearing the wire.

The two mechanisms can be combined if the corpuscules have an oval shape whose dimension according to the longitudinal axis allows the enclosure between two threads and its dimension according to the transverse axis, perpendicular to the axis of the wire, larger than the width of the thread, causes it to be embedded in the bone tissue.

It is important to underline an essential characteristic of the screw whose distal part must absolutely be tapered and conical so as to create between the core of the screw and the wall of the bone tunnel, a sufficiently wide space to allow the corpuscule to be trapped. It is easy to understand that a screw that is too large would inevitably imply the corpuscule to be driven out of the tunnel rather than being trapped in the wall of the tunnel. The same undesired effect would be caused if the screw were to meet corpuscules that were too large which would therefore be driven and pushed out by the screw, implying the collapse of the system. In order for the trapping to take place, the space between the tip of the screw and the wall of the tunnel must be at least as large or larger than the transverse diameter of the corpuscule. Thus, the screw can progress without driving the corpuscule out of the tunnel, but on the contrary it will trap it in the space located between itself (its core) and the wall of the bone tunnel. Once the tapered segment of the screw has overtaken the corpuscule, impacting it into the bone wall, any increase in the screw's diameter in its proximal segment will only increase the trapping depth of the corpuscule.

The invention shall become clearer after the reading of the following description, considering the appended drawings, provided solely by way of non-limiting example. The different structural elements of the invention and their arrangements are illustrated in these drawings wherein:

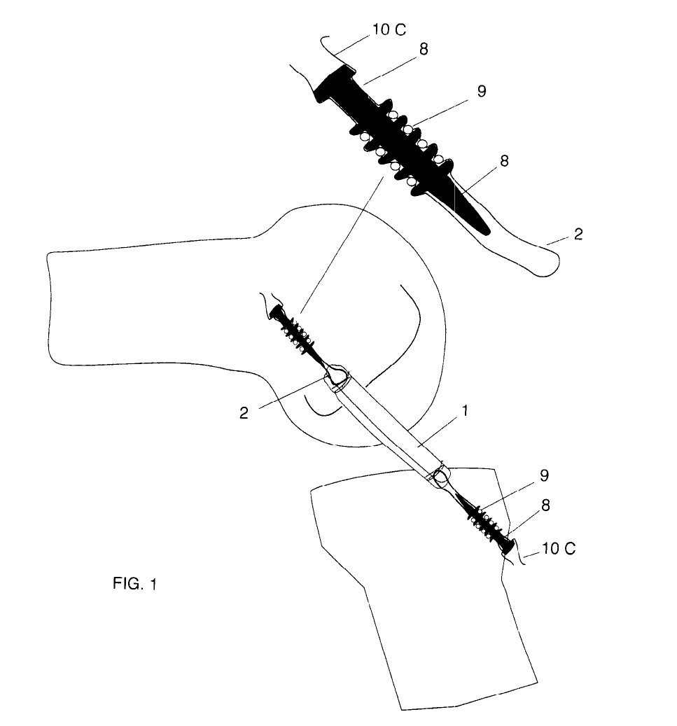

FIG. 1 schematically represents the final appearance of a graft with a screw 8, wire 10C (see FIG. 8) and corpuscules 9 having a spherical shape and a smaller diameter than the distance between two screw threads, in position on the femur-tibia assembly with an inset showing an enlargement of the screw 8.

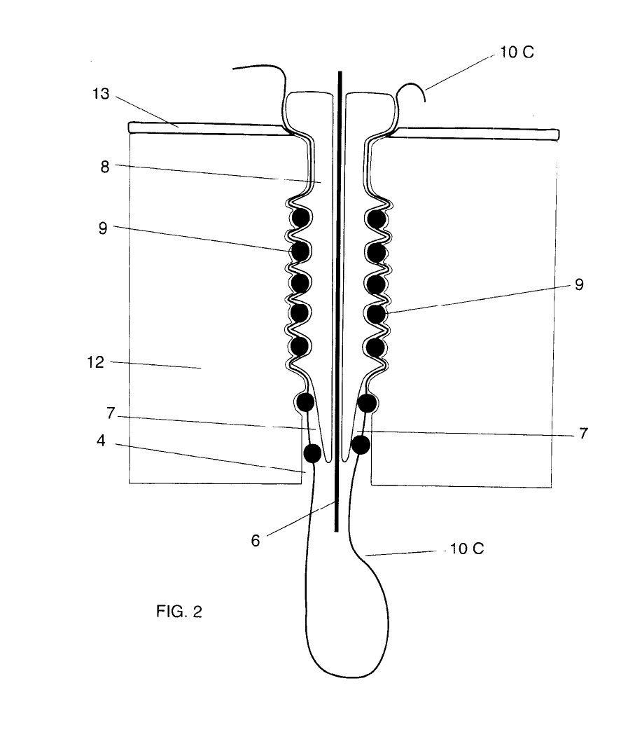

FIG. 2 represents a sectional longitudinal enlargement of a bone segment comprising its spongy part 12 and its cortical layer 13, crossed by a bone tunnel 4 in which a guide pin 6 and the suspension wire 10C equipped with its corpuscules 9 have been inserted. The screw 8, cannulated in its centre according to its longitudinal axis, has been threaded onto the guide pin 6 and then penetrates into the bone tunnel 4 according to the path imposed by the pin, crushing inside the spongy bone tissue 12 each corpuscule 9 that is thus trapped above and below by two adjacent screw threads and laterally by the core of the screw on one side and by the bone tissue on the other. It is to be noted that the space 7 located between the tapered distal portion of the screw and the wall of the tunnel allows the corpuscules to find a way in without being driven out by the body of the screw.

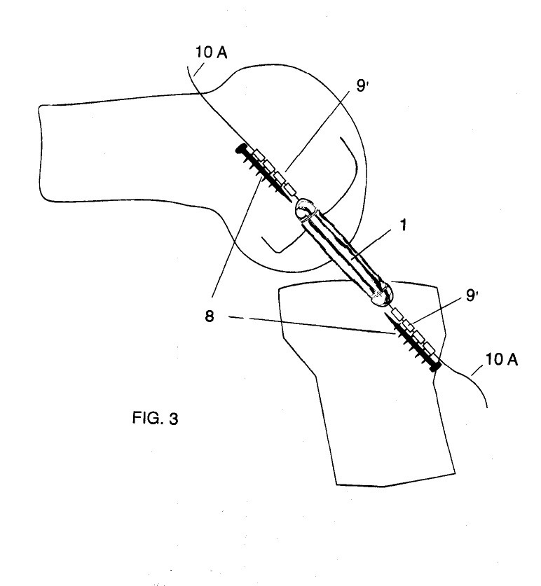

FIG. 3 schematically represents the final appearance of a graft 1 with a screw 8, by attaching the wire 10A equipped with oblong corpuscules 9′ in position on the femur-tibia assembly.

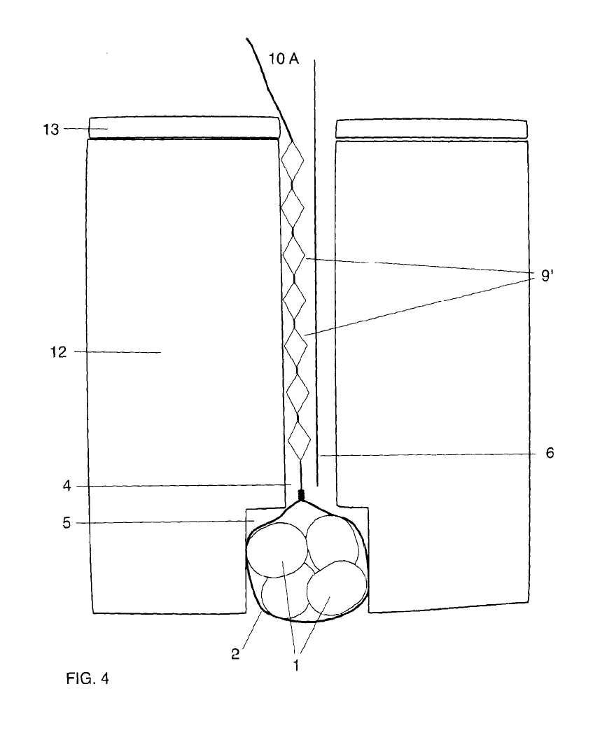

FIG. 4 represents a sectional longitudinal enlargement of a segment of spongy bone 12 and cortical bone 13 in which a bone tunnel 4 and a bone seat 5 have been realised. The wire 10A, provided with oblong rhomboid shaped corpuscules 9′ and a loop 2 suspending the 4-strand graft 1 (4 strand?) passes freely into the tunnel, from the inside to the outside (in the direction of its cortical orifice) and, by traction, draws the graft 1 behind it until the latter abuts the bottom of the seat housing 5. The guide pin 6 also passes freely into the bone tunnel 4.

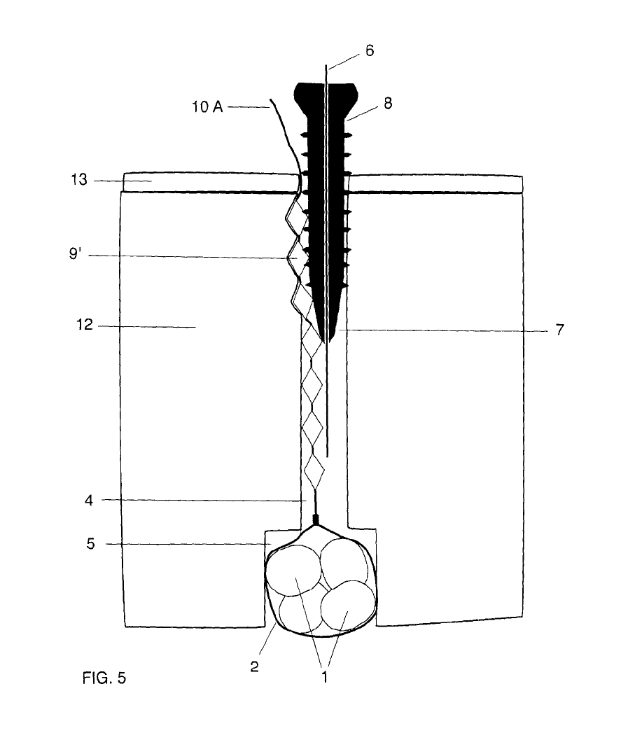

FIG. 5 shows the same components and illustrates the start of the insertion of the screw 8 which travels in the bone tunnel 4 according to the route imposed by the guide pin 6. It is to be noted that the space 7 resulting from the tapering of the distal segment of the screw allows it, during its progression in the tunnel, to “overtake” the corpuscule without driving it out of the tunnel, thus locking it in the space delimited by the overlying and underlying thread, the core of the screw and the nearby bone.

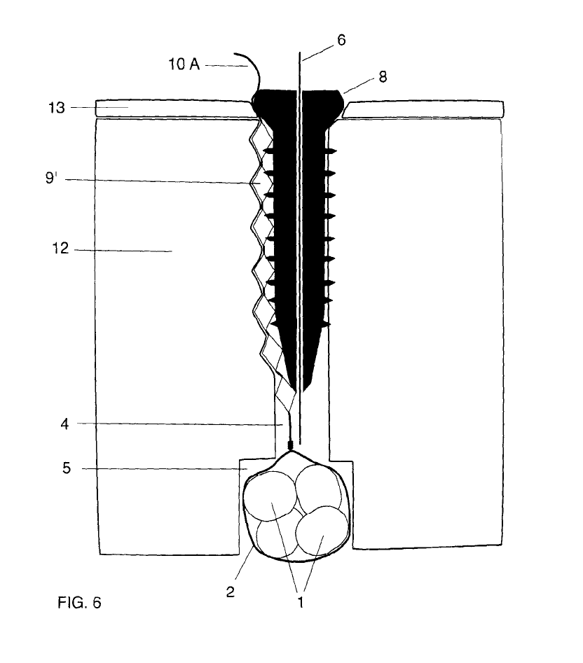

FIG. 6 shows the same components and illustrates the position of the corpuscules 9′, embedded in the spongy bone 12, after the complete insertion of the screw 8.

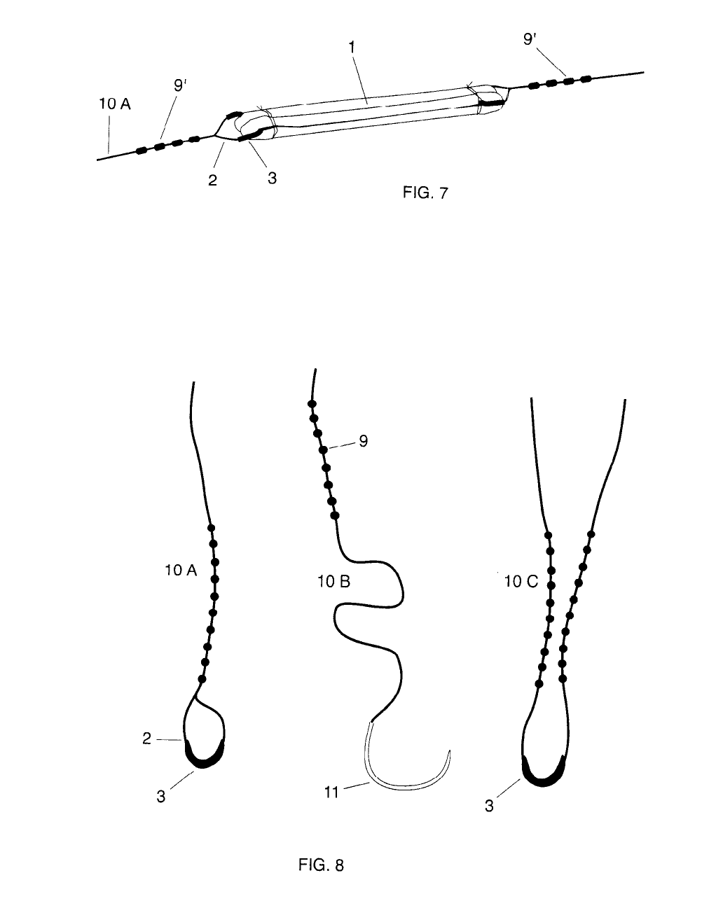

FIG. 7 shows a graft 1 suspended at each of its ends by the loop 2 equipped with an anti-shearing sheath 3 and a wire 10A provided with oblong corpuscules 9′.

FIG. 8 shows three embodiments of corpuscular wires which are illustrated herein purely by way of example, therefore without excluding any other embodiments that may be adapted to other applications. The suspension wire 10A is comprised of a single filament which finishes at one of its ends with a closed loop 2 intended to suspend the graft and possibly equipped with an anti-shearing sheath 3. The suspension wire 10B finishes with a crimped needle 11 which allows the lacing of the end of the soft tissue that is to be re-attached to the bone. The wire 10C is folded onto itself to form a natural loop intended to suspend the graft, each suspension strand being able to be equipped with corpuscules.

The anterior cruciate ligament reconstruction technique according to the invention is described below purely by way of example and therefore does not constitute an exclusive application of the device and process of the invention.

Preparation of the Graft.

The semitendinosus is typically harvested by means of a stripper.

As in the TLS technique, the tendon is prepared by rolling it onto itself to form a closed 4-strand short graft 1 allowing it to be suspended by means of a traction loop 2 arranged at each end of the graft. In the TLS technique, the ribbons pass freely through each end of the graft and can therefore be inserted after the graft has been realised.

In the present technique, in the event of use a wire type 10A, the loops 2 are closed and the graft must therefore realised by passing the tendon strands through the two loops already arranged on the preparation table. Since the diameter of the wire is very small (1 to 2 mm), a protective sheath 3 made of textile or composite material can be added to the loop of the wire (either during manufacture or at the time of use), so as to prevent any risks of the wire shearing the graft. Otherwise, like in the TLS technique, the tendon strands are fixed to each other by one to two suture stitches placed at each end of the graft.

Preparation of the bone tunnels and seats.

The tunnels and seats are prepared in the conventional way according to known techniques and left to the choice of the surgeon.

All these techniques always imply the placement of guide pins 6 on which cannulated drill bits or cutters slide, in order to bore the bone tunnels 4 and the seats 5 for inserting the graft 1. At the femur, the boring of the tunnels 4 and seats 5 is performed, according to the choice of the surgeon, either from the inside to the outside with the aid of in-out guides or with the aid of flexible pins or bits (which leave the surgeon to choose the insertion site that he deems best), or from the outside to the inside, using out-in guides. At the tibia, the placement of the guide pin 6 is made from the outside to the inside with the aid of suitable guides and the seat is performed backwards by means of specific tools available on the market.

The boring techniques of the tunnels 4 and seats 5 mentioned above do not constitute specific characteristics to the present invention except in relation to the diameter of the bone tunnels to be realised.

It is underlined herein that the conventional ligamentoplasty techniques impose, at the tibia, the boring of a bone tunnel having a slightly larger gauge than the size of the graft itself (about 8 to 11 mm). The TLS technique already allowed the gauge of the tunnels to be reduced to a diameter of 4.5 mm, necessary for the passage of the ribbons. The present technique allows the boring of a bone tunnel reduced to a final diameter of 2 to 3 mm and suppresses the need to realise a specific seat for receiving the attaching member as the TLS technique required, which implies a significant saving on bone tissue. Due to its very small dimensions, the “endobutton” type attachment, widely used in ligament surgery, is also able to pass though a small bone tunnel. Aside from the present invention, this is therefore the only one which currently allows a ligament graft to be attached by suspension through a small bone tunnel, both at the femur and at the tibia. However, the attachment obtained by this system is performed at femoral and tibial cortical level, therefore at a significant distance from the graft insertion site. It has now been clearly demonstrated that the inevitable elasticity of the textile bridges arranged between the graft and the cortical attaching member constitutes a considerable mechanical drawback, that jeopardises the quality of the ligament reconstruction. The present invention is therefore, to our knowledge, the only system that simultaneously has the three following functional properties:
fixing a ligament graft by suspension through very small bone tunnels (2 to 3 mm) both at the tibia and at the femur.—counteracting the elasticity of the attaching bridges until contact of the graft (which the TLS already did)
removing the need to bore a specific housing to receive the attaching member (which the TLS system required).

Insertion of the Graft

Before inserting the graft 1 into the knee then into the bone seats 5, care will have been taken to arrange a fine guide pin 6 in each of the femoral and tibial tunnels 4, making use of the skin of the thigh and the tibia, intended to guide further the route of the locking screw.

As in the TLS technique, the graft is inserted into the knee by the anteromedial approach. The suspension wire 10 of the femoral segment of the graft is passed into the femoral tunnel 2 through its intra-articular orifice and is recovered at the outer face of the femur by a cutaneous orifice of 2 or 3 mm, which the femoral guide pin also makes use of. The traction on this wire 10 causes the penetration of the graft 1 into the femoral seat 5 until it abuts onto the bottom of this housing. A similar operation is performed at the tibia for arranging the tibial insertion segment of the graft 1.

Locking the Graft

While maintaining the traction on the femoral wire, a conventional cannulated screw 8 is inserted onto the guide pin 6, normally used in orthopaedics, whose length is chosen according to the length of the segment of wire to be locked.

It has been noted that this operation is performed very easily, without having to surgically approach the outer surface of the bone concerned and therefore without having to create a cutaneous approach (except for the 2 or 3 mm necessary for the passage of the wire 10 and the guide pin 6).

It has also been noted that the present invention allows the risks of screwing defects inherent to the TLS system to be prevented (excess or insufficient insertion of the screw or even divergence of the screw in relation to the bone tunnel axis). In fact, the cannulated screw 8 follows a path that is necessarily parallel to the wire 10 thanks to the guide pin 6 ruling out all risks of divergence. On the other hand, thanks to the screw head which is automatically stopped at cortical level, the risk of excess or insufficient screwing is also entirely ruled out.

The same operation allows the locking of the graft to the tibia.

Although the present invention describes a system that is particularly suitable for the surgical reconstruction of the anterior cruciate ligament by short graft, it is not a choice that excludes the possibility to apply the system to other situations encountered in surgery. The same system is very well suited, for example, to posterior cruciate ligament reconstruction, internal and external lateral ligament reconstruction, but could also be used for any other procedure that requires the attachment of soft, tendon or ligament structures, to bone structures.

In a more general way, the present invention discloses a simple way of firmly attaching, by means of a cannulated or non-cannulated bone screw, all kinds of wires needing to be attached into a bone structure, thus being able to avoid having to resort to current sophisticated devices (intraosseous anchors etc.) often very expensive to use.

Another potential application of a simple wire provided with corpuscules at its two ends is also worthy of mention. It involves providing a protection system for the graft against excess tension throughout its whole healing step. In fact, a ligament graft remains fragile for many months after its realisation and is susceptible to stretching or even breaking if it is subjected too soon to tensile forces that are too high.

Upon the realisation of the graft, a simple wire could be arranged parallel thereto, provided with corpuscules at each of its ends, called “protector” wire, giving it a slightly longer length than that of the “suspension wire-graft-suspension wire” assembly. The suspension wire and the protector wire would be fixed at each end of the graft in the same bone tunnels by means of the same tibial and femoral screws. Upon any movement of the knee that causes an anterior displacement of the tibia in relation to the femur, the tension arises first inside the graft itself, the latter being shorter but, in the event of larger displacement, the tension then spreads to the protector filament, which automatically stops the risk movement, protecting the graft from excess tension or even the risk of breaking.

1.-13. (canceled)

14. A device for attaching soft tissue to a bone tissue, comprising:
a flexible wire provided with several corpuscules attached along its length, adapted to be introduced into a bone tunnel; and
a screw adapted to engage with said corpuscules of the wire upon its screwing into said bone tunnel locking it between the thread of the screw and the bone tissue.

15. The device according to claim 14, wherein the wire is adapted to form a free loop adapted to receiving said soft tissue, the two branches of the loop being equipped with said corpuscules.

16. The device according to claim 14, wherein the wire is equipped with or forms a closed loop at one of its ends, adapted to receive said soft tissue.

17. The device according to claim 14, wherein the wire finishes with a crimped needle, for example allowing the lacing of the end of the ligament that is to be re-attached to the bone.

18. The device according to claim 14, wherein the screw is a cannulated screw and also comprises a guide pin adapted to engage with the screw.

19. The device according to claim 14, wherein the screw has a distal tapered segment adapted to create a space sufficient to allow a corpuscule to find a way between it and the wall of the tunnel.

20. The device according to claim 14, wherein the screw has an increasing gauge towards the head of the screw.

21. The device according to claim 14, wherein the screw comprises a constant gauge section but of such a dimension that it laterally drives the corpuscule at least to come into contact with the bone wall or the inside thereof.

22. The device according to claim 14, wherein the corpuscules are smaller than or equal to the distance between two adjacent segments of screw threads, or oblong, having a longitudinal dimension greater than said distance.

23. The device according to claim 14, wherein the corpuscules are spherical, round or angular shaped, having a longitudinal dimension lower than the screw pitch so as to be able to be fixed and locked between two adjacent segments of the thread of the screw and with a transverse dimension such that they are adapted to be flush with the wall of the bone tunnel or to be embedded therein.

24. The device according to claim 14, wherein an extra wire is provided, intended to be arranged parallel to the graft but with a slightly longer length than the graft-attachment assembly and attached by the same screws and in the same bone tunnel as for attaching the graft.

25. The device according to claim 14 wherein, separately or in combination:
the corpuscules are distanced from each other by 2 to 20 mm;
the transverse dimension ranges from 1 to 3 mm and their longitudinal dimension from 2 to 8 mm; and
the diameter of the wire ranges from 0.5 to 3 mm, that of the screw from 3 to 8 mm.

26. A flexible wire provided with corpuscules along its length, for attaching soft tissue to a bone tissue for use in a device or system according to claim 14.

27. The device according to claim 26, wherein, separately or in combination:
the corpuscules are distanced from each other by 2 to 20 mm;
the transverse dimension ranges from 1 to 3 mm and their longitudinal dimension from 2 to 8 mm; and
the diameter of the wire ranges from 0.5 to 3 mm, that of the screw from 3 to 8 mm.
